(19) United States
(12) Patent Application Publication
(10) Pub. No.: US 2008/0153831 A1
(43) Pub. Date: Jun. 26, 2008

(54) ISOFLAVONE-CONTAINING COMPOSITIONS FOR THE TREATMENT OF OSTEOPOROSIS AND INFLAMMATORY JOINT DISEASE

(75) Inventors: Larry Dillaha, Atlanta, GA (US); Brian Adams, Atlanta, GA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Sciele Pharma, Inc.

(21) Appl. No.: 11/875,687

(22) Filed: Oct. 19, 2007

Related U.S. Application Data
(60) Provisional application No. 60/863,286, filed on Oct. 27, 2006.

(51) Int. Cl. A61K 31/519; A61P 3/00; A61P 29/00

(52) U.S. Cl. 514/249

(57) ABSTRACT

Compositions and methods for the prevention and treatment of osteoporosis are provided herein. The compositions contain one or more isoflavones, such as genistein and derivatives thereof, in combination with one or more folates, such as a reduced folate and/or folic acid; and/or one or more polyunsaturated fatty acids (PUFAs), such as an omega-3 and/or omega-6 fatty acid. The isoflavones, folates and/or essential fatty acid may be administered together or in separate dosage units. The compositions may optionally contain other vitamins, minerals, and ingredients, such as, emollient laxatives. The compositions are useful in the treatment of all forms of osteoporosis, including primary osteoporosis and secondary osteoporosis, and/or inflammatory joint diseases, especially in patients having a folic acid metabolism deficiency. The compositions are particularly useful in the treatment of inflammatory joint diseases, with complications that include bone loss, fracture, and osteoporosis. In addition, the compositions are beneficial for the prevention of osteoporosis in subjects who do not yet have the disease, but who are at risk for getting osteoporosis, such as post-menopausal women, subjects with osteopenia (mid thinning of the bone mass), subjects with an inflammatory joint disease, or people who are over the age of 70.

### CROSS REFERENCE TO RELATED APPLICATION

This application claims benefit of and priority to U.S. Provisional Patent Application No. 60/863,286 filed Oct. 27, 2006, and where permissible is incorporated by reference herein in its entirety.

### FIELD OF THE INVENTION

The present invention relates generally to compositions and methods for the treatment and prevention of osteoporosis and/or inflammatory joint disease.

### BACKGROUND OF THE INVENTION

Osteoporosis is defined as a reduction in bone mass and bone density with retention of the normal chemical composition of bone. More specifically, osteoporosis is a generalized, progressive diminution of bone density, i.e. bone mass per unit volume, causing skeletal weakness. Approximately 30 to 40% of the skeletal mass must be lost in order to reliably diagnose osteoporosis by radiology. Contemporary medicine distinguishes between primary and secondary osteoporosis (The Merck Manual of Diagnosis and Therapy, 17th ed., 1999). Primary osteoporosis includes juvenile osteoporosis, a rare form that occurs in children and young adults; Type I or postmenopausal osteoporosis, occurring in women between the ages of 50 and 75; and Type II or age-associated or senile osteoporosis, which usually occurs in men and women over 70 years of age. Primary osteoporosis is characterized by a predominant osteoclast activity and a disruption of the feedback mechanism between the serum calcium level and the parathyroid hormone (PTH) secretion and occurs uniformly throughout the whole skeleton. Secondary osteoporosis, accounting for less than 5% of all osteoporosis cases, results from chronic conditions that contribute significantly to accelerated bone loss. These conditions include endogenous and exogenous thyroxine excess, hyperparathyroidism, malignancies, gastrointestinal diseases, medications, renal failure and connective tissue diseases. Secondary osteoporosis usually begins in the main skeleton and progresses centrifugally.

Osteoporosis is characterized by pain in the respective bones; diffuse back pain; vertebral body collapse; and pathological fractures, in particular, fractures of the neck and femur. The goal of the management of all types of osteoporosis is therefore to decrease pain, to prevent fractures and to maintain body functions.

Osteoporosis is a common clinical feature and common complication in patients affected with chronic inflammatory diseases with joint manifestations. Examples of such diseases include rheumatoid arthritis (RA), juvenile rheumatoid arthritis (JRA), psoriatic arthritis, Reiter's syndrome (reactive arthritis), Crohn's disease, ulcerative colitis, and sarcoidosis (Orcel, P.; Cohen-Solal, M.; de Vernejoul, M. C., and Kuntz, D., Rev Rhum Mal Osteoartic. September 1992; 59(6 Pt 2):16S-22S; Brown, J. H. and Deluca, S. A., Am. Fam. Physician, 1995 October; 52(5);1372-80; De Vos, M.; De Keyser, F.; Mielants, H.; Cuvelier, C., and Veys, E., Aliment Pharmacol. Ther. 1998 May; 12(5):397-404; Falcini, F.; Trapani, S.; Civinini, R.; Capone, A.; Ermini, M., and Bartolozzi, G., J Endocrinol. Invest. March 1996; 19(3):165-9; and Scutellari, P. N. and Orzincolo, C., Eur. J. Radiol. 1998 May; 27 Suppl. 1:S31-8).

Rheumatoid arthritis is associated with a decrease in bone mass (Cortet, B.; Flipo, R. M.; Blanckaert, F.; Duquesnoy, B.; Marchandise, X., and Delcambre, B., Rev. Rhum. Engl. Ed. July 1997-Sep. 30, 1997; 64(7-9):451-8). Inflammatory arthritis often results in juxta-articular osteoporosis, cartilage loss, and cortical or marginal bone erosions (Lawson, J. P. and Steere, A. C. Lyme, Radiology, January 1985; 154(1):37-43; and Grassi, W.; De Angelis, R.; Lamanna, G., and Cervini, C., Eur. J. Radiol. May 1998; 27 Suppl 1:S18-24).

Joint inflammation exerts both local and systemic effects on skeletal tissues. Three forms of bone disease (bone loss) have been described in rheumatoid arthritis, namely: focal bone loss affecting the immediate subchondral bone and bone at the joint margins; periarticular osteopenia adjacent to inflamed joints; and generalized osteoporosis involving the axial and appendicular skeleton (Goldring, S. R. and Gravallese, E. M., Arthritis Res. 2000; 2(1):33-7). During chronic inflammatory joint diseases, such as rheumatoid arthritis, synovial cells produce large amounts of cytokines leading to increased local bone resorption and juxta-articular bone destructions (Orcel, P.; Cohen-Solal, M.; de Vernejoul, M. C., and Kuntz, D., Rev. Rhum. Mal. Osteoartic, September 1992; 59(6 Pt 2):16S-22S).

Homocysteinemia (the accumulation of homocysteine in plasma and tissue) is the result of deficiencies of certain enzymes and/or substrates involved in the transmethylation pathways. Homocysteinemia is caused by the accumulation of homocysteine and its two disulfides in plasma and tissue (Mudd et al., The Metabolic Basis of Inherited Disease, New York, McGraw-Hill, 1978, p. 458). Homocysteinemia is associated with juvenile arteriosclerosis, recurrent arterial and venous thromboembolic manifestations and osteoporosis. The latter may be due to the fact that homocysteine also interferes with collagen synthesis, and it is this interaction that may be significant in the development of defective bone matrix and osteoporosis (Am. J. Med. Sci., 273, 1977, p. 120). In addition, studies have concluded that an increased homocysteine level appears to be a strong and independent risk factor for osteoporotic fractures (van Meurs et al., N. Engl. J. Med. 2004 May 13; 350(20);2033-41; Cashman, K D., Nutr. Rev. 2005 January; 63(1):29-36). Folio acid has been described as a successful tool for the treatment of hyperhomocysteinemia (Brattstrom et al., Metabolism, Vol. 34, No. 11, 1985, p. 1073). For this reason, folic acid has been included in compositions to treat osteoporosis, for example, as described in U.S. Pat. Nos. 6,790,462 to Hendricks; 6,881,419 to Lovett; and 4,902,718 to Bayless et al.

There is a drawback to these compositions because the biologically active form of folic acid, 5-methyltetrahydrofolate (5-MTHF), may not be effective in some patients due to a common genetic mutation. For example, a study published in 2000 by Botto and Yang of the Centers for Disease Control and Prevention, in the American Journal of Epidemiology (Botto, L. D., and Yang, Q. American Journal of Epidemiology, Vol 151, Issue 9: 862-877) demonstrated that one in eight women have a genetic trait that can prevent proper metabolism of folic acid. The trait is classified as homozygosity for the T allele of the C677T polymorphism of the gene encoding the folate dependent enzyme 5,10-methylenetetrahydrofolate reductase (MTHFR). It was reported by Botto and Yang that the homozygous genotype can be present in more than 40 percent of Hispanic women. The homozygous genotype was also observed in other ethnic subgroups. In a study by Peng, F., Labelle, L. A., Rainey, B. J., Tsongalis, G. J., Int. J. Mol. Med. 2001; 8: 509-511, the prevalence of a C677T or A1298C single nucleotide polymorphism (SNP) was investigated. Homozygosity for the C677T MTHFR SNP was detected in 16% and 10% of Caucasians and Hispanics, respectively. The frequency of the C677T heterozygous SNP for Caucasians and Hispanics was 56% and 52%, respectively. Because of the inability of some individuals to properly metabolize folic acid, there is a need for improved folic-acid containing compositions for the treatment of osteoporosis.

The use of polyunsaturated fatty acids (PUFAs) in the prevention of postmenopausal osteoporosis has been reviewed in the literature (Albertazzi and Coupland, Maturitas, Vol. 42, No. 1, 13-22 (2002)). PUFAs are the starting material for the production of eicosanoids, which include prostaglandins, thromboxane, and leukotrienes. The two essential fatty acid families (n-3 and n-6) are converted into two distinct families of eicosanoids each with unique physiological properties. Competition between the two classes of PUFAs occurs in prostaglandin formation. The activity of bone formation and bone resorption is regulated by synthetic hormones and local factors produced in bone. Of the local factors that act on bone cells, eicosanoids appear to be the principle mediator. Prostaglandin E2 (PGE₂) is a potent stimulator of bone resorption and the primary prostaglandin affecting bone metabolism. Prostaglandins also affect the synthesis and action of insulin-like growth factors that are major bone-derived growth factors. PGE₂ was reported to increase cortical bone mass and intracortical bone remodeling in both intact and ovariectomized rats and increase proximal tibial metaphyseal bone area in osteopenic ovariectomized rats.

It is therefore an object of the present invention to provide improved compositions for the treatment and/or the prevention of osteoporosis and/or inflammatory joint diseases.

It is another object of the invention to provide improved compositions for the treatment and/or the prevention of osteoporosis and/or inflammatory joint diseases in subjects with a folic acid metabolism deficiency.

### SUMMARY OF THE INVENTION

Compositions and methods for the prevention and treatment of osteoporosis and/or inflammatory joint diseases are provided herein. The compositions contain one or more isoflavones, such as genistein and derivatives thereof, in combination with one or more folates, such as a reduced folate and/or folic acid; and/or one or more polyunsaturated fatty acids (PUFAs), such as an omega-3 and/or omega-6 fatty acid. The isoflavones, folates and/or essential fatty acids may be administered together or in separate dosage units. In one embodiment, the reduced folate is a 5-methyltetrahydrofolate, such as 5-methyl-(6S)-tetrahydrofolic acid and the polyunsaturated fatty acid is an omega-3 fatty acid, containing docosahexenoic acid (DHA), eicosapentaenoic acid (EPA), or mixtures thereof. The compositions may optionally contain other vitamins, minerals, and ingredients, such as emollient laxatives. In another embodiment, the composition contains a form of zinc.

The compositions may be administered to treat all forms of osteoporosis, including primary osteoporosis and secondary osteoporosis, and/or inflammatory joint diseases, especially in patients having a folic acid metabolism deficiency. The compositions may also be administered to treat inflammatory joint diseases with complications that include bone loss, fracture, and osteoporosis. In addition, the compositions may be administered for the prevention of osteoporosis in subjects who do not yet have the disease, but who are at risk for developing osteoporosis, such as post-menopausal women, subjects with osteopenia (mild thinning of the bone mass), subjects with an inflammatory joint disease, or people who are over the age of 70.

### DETAILED DESCRIPTION OF THE INVENTION

### I. Definitions

As used herein, the term “one or more folates” includes (1) folic acid, (2) the anionic form of folic acid, folate; and (3) natural and synthetic isomers of reduced folate or pharmaceutically acceptable salts thereof, and combinations thereof.

As used herein, “reduced folate” includes both natural and synthetic isomers of reduced folate.

As used herein, “one or more polyunsaturated fatty acids” (PUFAs) refers to one or more fatty acids that contain more than one site of unsaturation (e.g. double bond) including, but not limited to, omega-3 fatty acids including, but not limited to, eicosapentaenoic acid (EPA), docosahexaenoic acid (DHA) and alpha-linolenic acid (ALA); omega-6 fatty acids including, but not limited to, linoleic acid (LA) and arachidonic acid (AA); omega-9 fatty acids; and mixtures thereof.

As used herein, “an effective amount” or “a therapeutically effective amount” means an amount sufficient for the prevention, inhibition, treatment, or amelioration, or otherwise beneficial alteration of one or more of the symptoms of osteoporosis, osteopenia and/or an inflammatory joint disease.

As used herein, “bioavailable” and “bioavailability” are interchangeable and refer to the degree to which, or rate at which, a drug or other substance is absorbed or becomes available at the site of physiological activity after administration.

As used herein, “substantially free of other polyunsaturated fatty acids” means less than 10% by weight, preferably less than 5% by weight of the other polyunsaturated fatty acids are present in the composition.

As used herein, “emollient laxative” means a stool softener.

As used herein, “dosage unit” refers any pharmaceutically acceptable form for administering the composition to a patient. The drug can be administered enterally, parenterally, or topically. In one embodiment, the composition is administered orally, Suitable oral dosage units include, but are not limited to, capsules, including, but not limited to hard or soft gelatin or non-gelatin capsules; tablets; buccal forms; troches; lozenges; liquids; suspensions; solutions; or emulsions.

As used herein, “defective folate metabolic pathway”, “deficient folic acid metabolic pathway” or “folic acid metabolism disorder” refers to a lower than normal, lack of, inhibited, or restricted production of folic acid pathway metabolites. These terms also refer to lower than normal, deficient or defective levels of folic acid metabolites in a human or other animal.

### II. Compositions

The compositions contain therapeutically effective amounts of one or more isoflavones in combination with one or more folates and/or one or more polyunsaturated fatty acids (PUFAs).

The compositions optionally contain a therapeutically effective amount of one or more additional ingredients such as zinc, iron, copper, vitamin D, vitamin B₁, vitamin B₂, vitamin B₁₂, vitamin B₆, vitamin E, vitamin C, biotin, panthothenic acid, niacinamide, vitamin A, magnesium, calcium, and emollient laxatives. In one embodiment, the formulation includes zinc.

A. Isoflavones

Isoflavones are a class of compounds known to increase bone density. These compounds are available from a variety of dietary sources, such as soy, kudzu, fava beans, and lupines. Isoflavones appear to be widely distributed in the plant kingdom and over 700 different isoflavones are known and characterized. Suitable isoflavones include, but are not limited to, those described in U.S. Pat. Nos. 6,495,141 to Waggle et al.; 6,146,668 Kelly et al.; and 6,391,309 to Empie et al. In one embodiment, the isoflavones include those that are distinguished by their ability to bind to estrogen receptors on animal (including human) cells, including, but not limited to, daidzein, genistein, biochanin A, formononctin and glycitein in a variety of forms (e.g., glycosidic and acetylated forms). Soy isoflavones are commercially available, for example, from Archer-Daniels-Midland Co. of Decatur, Ill. Synthetically derived isoflavones may also be used. The compositions contain one or more isoflavones, or derivatives thereof, in a concentration so that the daily consumption is in the range of from about 1 mg to about 500 mg, preferably from about 5 mg to about 100 mg.

B. Folates

The isoflavone-containing compositions described above may contain one or more folates.

i. Reduced Folates

Reduced folates and compositions containing these compounds are well-known and described in the art, for example, in U.S. Pat. Nos. 5,350,851 and 5,997,915 to Bailey et al.; 6,011,040 and 6,441,168 to Muller et al.; and 6,921,754 to Hainlein et al. Natural isomers of reduced folate suitable for use in the compositions include, but are not limited to, (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid. Other natural isomers of reduced folate include the polyglutamyls, such as the diglutamyl, triglutamyl, tetraglutamyl, pentaglutamyl, and hexaglutarnyl, derivatives of (6S)-tetrahydrofolic acid, 5-methyl-(6S)-tetrahydrofolic acid, 5-formyl-(6S)-tetrahydrofolic acid, 10-formyl-(6R)-tetrahydrofolic acid, 5,10-methylene-(6R)-tetrahydrofolic acid, 5,10-methenyl-(6R)-tetrahydrofolic acid, and 5-formimino-(6S)-tetrahydrofolic acid. The natural isomers may be administered in combination with one or more synthetic isomers of reduced folate, such as (6R)-tetrahydrofolic acid, 5-methyl-(6R)-tetrahydrofolic acid, 5-formyl-(6S)-tetrahydrofolic acid, 10-formyl-(6S)-tetrahydrofolic acid, 5,10-methylene-(6S)-tetrahydrofolic acid, 5,10-methenyl-(6S)-tetrahydrofolic acid, 5-formimino-(6R)-tetrahydrofolic acid, and polyglutamyl derivatives thereof. The natural and synthetic folates may be present in equal amounts or different amounts. Any or all of the natural and synthetic isomers of reduced folate can be present as a single enantiomer or a mixture of enantiomers and/or diastereomers.

In one embodiment, the reduced folate is 5-methyltetrahydrofolate. “5-methyltetrahydrofolate”, as used herein, refers to the compound N-(5-methyl)-5,6,7,8-tetrahydropteroyl)-L-glutamic acid or a pharmaceutically acceptable salt thereof. This compound is described in U.S. Pat. No. 5,997,915 to Bailey et al. Salt forms of this compound are described in U.S. Pat. No. 6,441,168 to Muller et al. In one embodiment, the 5-methyltetrahydrofolate is 5-methyl-(6S)-tetrahydrofolic acid.

Unlike folic acid, 5-methyltetrahydrofolate does not require enzymatic conversion to the biologically active compound. This enzymatic conversion process can be difficult for some individuals, especially those who carry a folate metabolic gene mutation. The population at risk, and the population that can benefit from the presence of 5-methyltetrahydrofolate supplementation, is much larger than previously believed. In addition, those individuals affected by a genetic mutation in the folate metabolic pathway, especially those mutations that affect 5-methyltetrahydrofolate production or function, can be aided through the administration of a composition comprising 5-methyltetrahydrofolate. Therefore, compositions comprising 5-methyltetrahydrofolate and other reduced folates, may eliminate, reduce or lessen the consequences of 5-methyltetrahydrofolate genetic deficiencies associated with folate metabolism.

5-methyltetrahydrofolate has been shown to be an ingredient of high bioavailability. Preliminary research suggests that 5-methyltetrahydrofolate is as bioavailable as folic acid. In particular circumstances, host-related factors, such as gastrointestinal illness and pH of the small intestine, can influence the bioavailability of folic acid, because it can be converted into the active form prior to transport across the blood-brain barrier. Compositions and nutritional preparations containing reduced folate in combination with folic acid are expected to more beneficial than those containing folic acid alone.

In one embodiment, the amount of reduced folate in the composition is from about 400 μg to about 7 mg. In another embodiment, the amount of reduced folate ranges from about 400 μg to about 4 mg. In a further embodiment, the composition contains 5-methyl-(6S)-tetrahydrofolic acid and the amount is from about 0.5 mg to about 2 mg. In yet another embodiment, the reduced folate is administered in a dose of from 400 μg to 7 mg, and preferably from 0.8 mg to 4 mg. In a preferred embodiment, the reduced folate is 5-methyl-(6S)-tetrahydrofolic acid administered in a dose of from about 0.8 mg to about 2 mg.

ii. Folic Acid

The compositions may also contain folic acid, or the anionic form of folic acid, folate. Compositions containing, for example, both the reduced folate, 5-methyltetrahydrofolate, and folic acid have the increased benefit of providing a readily available form of biologically active 5-methyltetrahydrofolate while simultaneously providing a longer term source of folate and folic acid. As discussed above, folic acid must undergo enzymatic conversion to the biologically active form. Therefore, the combination of 5-methyltetrahydrofolate and folic acid provides a longer term source of folates than the use of 5-methyltetrahydrofolate or another reduced folate alone, for example, as described in U.S. Pat. No. 6,812,215 to Buchholz et al. Therapeutically effective amounts of folic acid or folate in the compositions preferably range from about 50 μg to about 7 mg. In another embodiment, the amount of folic acid or folate is from about 400 μg to about 6 mg, preferably from about 1 mg to about 5 mg, more preferably from about 3 mg to about 5 mg. In a preferred embodiment, the amount of folic acid or folate is from about 1 mg to about 3 mg.

The “total amount of folate” provided in the compositions can be represented as the sum of (1) the folic acid; (2) the anionic form of folic acid, folate; and (3) the reduced folate. In one embodiment, the total amount of folate present in the compositions ranges from about 0% to about 40% reduced folate, and from about 60% to about 100% folic acid. In one embodiment, the total amount of folate in the compositions ranges from about 400 μg to about 7 mg. In a preferred embodiment, the total amount of folate in the compositions is greater than 0.8 mg, preferably from about 1 mg to about 5 mg, and more preferably from about 1 mg to about 3 mg.

C. Polyunsaturated Fatty Acids

The compositions optionally contain one or more polyunsaturated fatty acids (PUFAs), such as omega-3, omega-6, or omega-9 fatty acids, or mixtures thereof. PUFAs are well-known and their use in the treatment of osteoporosis is described, for example, in U.S. Patent Application Publication Nos. 20040082523 to Krammer et al. and 20020198177 to Horrobin. Alpha-linolenic (ALA), docosahexaenoic (DHA), and eicosapentaenoic (EPA) acids are examples of omega-3 fatty acids. Linoleic acid (LA) and arachidonic acid (AA) are examples of omega-6 fatty acids. Oleic (OA) and erucic acid (EA) are examples of omega-9 fatty acids. The omega-3 and omega-6 fatty acids are polyunsaturated fatty acids classified as essential because humans cannot synthesize fatty acids and must obtain them through the diet.

In one embodiment, the PUFA is an omega-3 fatty acid, or a mixture of omega-3 fatty acids, and preferably contains docosahexaenoic acid (DHA). DHA is one of the main components of brain and heart tissue. It is required for the proper functioning of all neural systems, including the brain, the retina and the central nervous system. DHA and vitamin/mineral compositions containing this essential fatty acid are described in detail in U.S. Patent Publication No. 2003/0050341 to Bydlon et al.

i. DHA Raw Material

DHA raw materials contain DHA, optionally with one or more additional PUFAs. DHA is mainly available as a fish oil extract, however, it may be desirable to use DMA derived from another natural source, such as algae (e.g. Crypthecodinium cohnii). Methods for the production of DHA from algae are described in the following patents, U.S. Pat. Nos. 5,130,242; 5,340,742; 5,340,594; and 6,451,567 to Barclay; 6,509,178 to Tanaka et al.; and 6,607,900 to Bailey.

In one embodiment, the compositions may contain a DHA raw material that is substantially free of other omega-3 fatty acids. In another embodiment, the DHA raw material is substantially free of omega-6 fatty acids, such as linoleic acid. In one embodiment the compositions contain a DHA raw material containing linoleic acid in concentrations less than or equal to 5% by weight by weight of the DHA raw material. In another embodiment, the DHA raw material contains at least about 40% by weight DHA relative to all other fatty acids. Therapeutically effective amounts of PUFAs that may be used in the compositions and preparations are from about 100 mg to about 1 g. In one embodiment, the amount of the PUFA present in the compositions and preparations ranges from about 200 mg to about 800 mg. In one embodiment, the PUFA is DHA in an amount from about 250 mg to about 500 mg. In another embodiment, the composition is in the form of a soft gelatin capsule and contains a DHA raw material that is substantially free of other vitamins, minerals and omega-3 fatty acids. In another embodiment, the softgel capsule may contain a DHA raw material that is substantially free of eicosapentaenoic acid and linolenic acid.

D. Additional Vitamins, Minerals, and Ingredients

The compositions described herein optionally contain one or more vitamins, mineral, nutriceuticals, or combinations thereof: Examples of vitamins, mineral and nutriceuticals include, but are not limited to, zinc, iron, copper, vitamins A, B₁, B₂, B₁₂, B₆, D, E, and C; biotin, pantothenic acid, niacinamide, magnesium, calcium and emollient laxatives.

i. Zinc

In one embodiment, the compositions contain a form of zinc (e.g. a zinc-containing compound or a derivative thereof). The advantageous effect of combining zinc, with an isoflavone, for the treatment of osteoporosis is described in U.S. Pat. No. 5,935,996 to Yamaguchi. When zinc is present in the compositions, the compositions typically contain from about 5 to about 100 mg of a zinc compound or a derivative thereof. In one embodiment, the amount of zinc in the compositions is from about 10 mg to about 30 mg. In another embodiment, the amount of zinc in the compositions is from about 12 mg to about 20 mg. In a preferred embodiment, the zinc compound is a zinc salt, such as zinc sulfate.

ii. Iron

The compositions optionally include an iron compound or derivatives thereof. In one embodiment, the amount of iron in the compositions ranges from about 10 mg to about 200 mg of iron compound or derivative thereof. In one embodiment, the iron compound is elemental iron. In another embodiment, the iron compound is carbonyl iron in a concentration from about 80 mg to about 130 mg, preferably about 90 mg. In yet another embodiment, the iron compound is an iron salt or combinations thereof including, but not limited to, ferrous sulfate, ferrous fumarate, ferrous succinate, ferrous gluconate, ferrous lactate, ferrous glutamate or ferrous glycinate in a concentration from about 20 mg to about 80 mg.

iii. Copper

The compositions optionally include a copper compound or derivatives thereof. Typically, the amount of copper in the compositions is from about 0.1 mg to about 10 mg of copper compound or derivative thereof. In one embodiment, the amount of copper in the compositions is from about 1 mg to about 5 mg. In a preferred embodiment, the amount of copper in the compositions is from about 1.5 mg to about 2.5 mg. In one embodiment, the copper compound is cupric oxide.

iv. Vitamin D

The compositions optionally contain vitamin D₃ (cholecalciferol) or a derivative thereof. Derivatives of vitamin D₃ include compounds formed from vitamin D₃ that are structurally distinct from vitamin D₃, but retain the active function of vitamin D₃. The amount of vitamin D₃ in the compositions typically ranges from about 1 IU to about 2000 IU. In one embodiment, the amount of vitamin D₃ in the compositions ranges from about 200 IU to about 1500 IU. In a preferred embodiment, the amount of vitamin D₃ in the compositions ranges from about 300 IU to about 1000 IU.

v. Vitamin B₁

The compositions optionally contain vitamin B₁ (thiamine mononitrate) or a derivative thereof. Derivatives of vitamin B₁ include compounds formed from vitamin B₁ that are structurally distinct from vitamin B₁, but retain the active function of vitamin B₁. The amount of vitamin B₁ in the compositions is from about 0.5 mg to about 50 mg. In one embodiment, the amount of vitamin B₁ in the compositions is from about 1 mg to about 4 mg. In a preferred embodiment, the amount of vitamin B₁ in the compositions is from about 2 mg to about 3.5 mg.

vi. Vitamin B₂

The compositions optionally include vitamin B₂ (riboflavin) or a derivative thereof. Derivatives of vitamin B₂ include compounds formed from vitamin B₂ that are structurally distinct from vitamin B₂, but retain the active function of vitamin B₂ The amount of vitamin B₂ in the compositions is typically from about 0.5 μg to about 50 mg. In one embodiment, the amount of vitamin B₂ typically in the compositions is from about 1 mg to about 4.5 mg. In a preferred embodiment, the amount of vitamin B₂ in the compositions is from about 3.0 mg to about 3.8 mg.

vii. Vitamin B₆

The compositions optionally contain vitamin B₆ (pyridoxine) or a derivative thereof. Derivatives of vitamin B₆ include compounds formed from vitamin B₆ that are structurally distinct from vitamin B₆, but retain the active function of vitamin B₆. The vitamin B₆ may be present in a single form or in various different forms in combination within the present compositions. The amount of vitamin B₆ in the compositions preferably ranges from about 0.1 mg to about 200 mg. In one embodiment, the amount of vitamin B₆ in the compositions ranges from about 2 mg to about 90 mg. In a preferred embodiment, the amount of vitamin B₆ in the compositions ranges from about 10 mg to about 50 mg.

viii. Vitamin B₁₂

The compositions optionally include a vitamin B₁₂ or one of the three active forms: cyanocobalamin, hydroxocobalamin, or nitrocobalamin, or derivatives thereof. Derivatives of vitamin B₁₂ include compounds formed from vitamin B₁₂ that are structurally distinct from vitamin B₁₂, but retain the active function of vitamin B₁₂. Non-limiting examples of such derivatives include methylcobalamin, deoxyadenosylobalamin, or combinations thereof. The amount of vitamin B₁₂ in the composition is typically from about 2 μg to about 250 μg. In one embodiment, the amount of vitamin B₁₂ in the compositions ranges from about 5 μg to about 30 μg. In a preferred embodiment, the amount of vitamin B₁₂ in the compositions ranges from about 10 μg to about 20 μg.

ix. Vitamin E

The composition optionally include vitamin E (dl-α-tocopheryl acetate) or a derivative thereof. Derivatives of vitamin E include compounds formed from vitamin E that are structurally distinct from vitamin E, but retain the active function of vitamin E. The amount of vitamin E in the compositions is typically from about 1 international unit (IU) to about 910 IU. In one embodiment, the amount of vitamin E in the compositions is from about 5 IU to about 500 IU. In a preferred embodiment, the amount of vitamin E in the compositions is from about 8 IU to about 200 IU.

x. Vitamin C

The compositions described herein may optionally include vitamin C (ascorbic acid) or a derivative thereof. Derivatives of vitamin C include compounds formed from vitamin C that are structurally distinct from vitamin C, but retain the active function of vitamin C. The amount of vitamin C in the compositions is typically from about 10 mg to about 2000 mg. In one embodiment, the amount of vitamin C in the compositions is about 75 mg to about 1000 mg. In a preferred embodiment, the amount of vitamin C in the compositions is from about 100 mg to about 500 mg.

xi. Biotin

The compositions optionally contain biotin or a derivative thereof. Derivatives of biotin include compounds formed from biotin that are structurally distinct from biotin, but retain the active function of biotin. The amount of biotin in the compositions is typically from about 10 μg to about 50 μg. In one embodiment, the amount of biotin in the compositions is from about 20 μg to about 40 μg. In a preferred embodiment, the amount of biotin in the compositions is from about 25 μg to about 35 μg.

xii. Pantothenic Acid

The compositions optionally include pantothenic acid (calcium pantothenate) or a derivative thereof. Derivatives of pantothenic acid include compounds formed from pantothenic acid that are structurally distinct from pantothenic acid, but retain the active function of pantothenic acid. The amount of pantothenic acid in the compositions is typically from about 1 mg to about 10 mg. In one embodiment, the amount of pantothenic acid in the compositions is from about 3 mg to about 8 mg. In a preferred embodiment, the amount of pantothenic acid in the compositions is from about 5 mg to about 7 mg.

xiii. Niacinamide

The compositions optionally include niacinamide or derivatives thereof. Derivatives of niacinamide include compounds formed from niacinamide that are structurally distinct from niacinamide, but retain the active function of niacinamide. The amount of niacinamide in the compositions is typically from about 1 mg to about 100 mg. In one embodiment, the amount of niacinamide in the compositions is from about 10 mg to about 30 mg. In a preferred embodiment, the amount of niacinamide in the compositions is from about 15 mg to about 25 mg.

xiv. Vitamin A

The compositions optionally include vitamin A from any commonly known source, for example, retinol or beta-carotene. Preferably, the source of vitamin A is beta-carotene. In one embodiment, vitamin A is provided in a total daily dose of between 0-10,000 I.U, and preferably between 2,000 and 5,000 IU.

xv. Magnesium

The compositions optionally include a magnesium compound or a derivative thereof. The amount of magnesium in the compositions typically ranges from about 5 mg to about 400 mg of a magnesium compound or a derivative thereof. In one embodiment, the amount of magnesium in the compositions ranges from about 10 mg to about 200 mg. In another embodiment, the amount of magnesium in the compositions ranges from about 20 mg to about 100 mg. In a preferred embodiment, the magnesium compound is magnesium oxide. Biologically-acceptable magnesium compounds that may be incorporated include, but are not limited to, magnesium stearate, magnesium carbonate, magnesium oxide, magnesium hydroxide and magnesium sulfate.

xvi. Calcium

The compositions optionally include a calcium compound or a derivative thereof. Because most of the body's calcium is found in the bones, an adequate intake of calcium is important to maximize and maintain bone density. A calcium-poor diet is a primary risk factor for osteoporosis. Calcium, combined with vitamin D, can help maintain or reduce the rate of bone loss that occurs with osteoporosis.

The amount of calcium compound or derivative thereof in the compositions ranges from about 20 mg to about 2500 mg. In one embodiment, the amount of calcium in the compositions is in excess of 200 mg and ranges from about 250 mg to about 2000 mg. In another embodiment, the amount of calcium in the compositions ranges from about 500 mg to about 1000 mg. In another embodiment, calcium is administered in a dose of from about 250 mg to about 2000 mg, and preferably from about 500 mg to about 1000 mg. Biologically-acceptable calcium compounds include, but are not limited to, any of the well known calcium supplements, such as calcium carbonate, calcium sulfate, calcium oxide, calcium hydroxide, calcium apatite, calcium citrate-malate, bone meal, oyster shell, calcium gluconate, calcium lactate, calcium phosphate, calcium levulinate, and the like. In a preferred embodiment, the calcium compound is calcium carbonate.

xvii. Emollient Laxatives

In one embodiment, the compositions include an emollient laxative. Suitable emollient laxatives include, but are not limited to, sodium docusate, calcium doucsate, glycerin, mineral oil or a poloxamer. The amount of emollient laxative is typically between about 50 mg and about 1 g. In one embodiment, the amount of emollient laxative in the composition is from about 50 to about 200 mg.

Although described above with reference to specific compounds, one can also utilize enantiomers, stereoisomers, polymorphs, derivatives and/or salts of the active compounds. Methods for synthesis and/or isolation of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418).

### III. Formulations

In one embodiment, the soft gelatin capsules (also referred to as “softgels”) are prepared from vegetable or plant based materials. In another embodiment, the soft gelatin capsules are made from cellulosic raw materials. The soft gelatin capsules may be preservative-free, easy to swallow, effectively mask taste and odor, and allow product visibility. Softgels may be prepared, for example, by dispersing the formulation in an appropriate vehicle to form a high viscosity mixture. This mixture is then encapsulated with a gelatin or vegetable based material using technology and machinery known to those in the softgel industry.

Film coated tablets, for example, may be prepared by coating tablets using techniques including, but not limited to, rotating pan coating methods or air suspension methods to deposit a contiguous film layer on a tablet. This procedure is often done to improve the aesthetic appearance of tablets, but may also be done to improve the ease of swallowing of tablets, or to mask an odor or taste. The compositions may conveniently be presented in unit dosage form and may be prepared by conventional pharmaceutical techniques. The compositions may be provided in a blister-pack or other such pharmaceutical package, without limitation

Preferably, the compounds are orally administered. For oral administration, the compounds are formed into tablets, granules, powders or capsules containing suitable amounts of granules or powders by a conventional method together with usual drug additives. Oral formulations containing the active compounds may be in any conventionally used oral form including, but not limited to, tablets, capsules, buccal forms, troches, lozenges and oral liquids, suspensions solutions, or emulsions. Oral formulations may utilize standard delay or time release formulations to alter the absorption of the active compound(s).

The one or more isoflavones, one or more folates and/or one or more polyunsaturated fatty acids may be contained within the same dosage unit or in separate dosage units. In one embodiment, they are contained in the same dosage unit. The preferred, but optional, ingredients can be provided (1) in the same dosage unit as the reduced folate and folic acid if the reduced folate or folic acid are formulated together, (2) in the same dosage unit as either the reduced folate or folic acid if the reduced folate or folic acid are provided in separate dosage units or (3) in a completely separate dosage unit from both the folic acid and the reduced folate. In one embodiment, the preferred ingredients are formulated in the same dosage unit as the reduced folate and folic acid 1n another embodiment, the reduced folate and folic acid and optional ingredients are formulated for administration in a single dosage unit, such as a tablet or capsule.

For example, the PUFAs can be contained within the same dosage unit as the reduced folate or folic acid, and optional ingredients may be in a separate dosage unit. In one embodiment, the PUFAs are provided in a separate dosage unit that is substantially free of other vitamins or minerals. The PUFA may be in a capsule. For example, the compositions can contain at least one tablet containing a reduced folate and folic acid and at least one softgel capsule containing EFAs packaged together in a single product. In one embodiment, a PUFA is encapsulated in a semi-solid or liquid form. In another embodiment, the PUFA is DHA or mixture of polyunsaturated fatty acids containing DHA, which is presented in a semi-solid or liquid form packaged in a soft gelatin capsule.

By way of further example, ingredients such as the one or more isoflavones, folic acid, or reduced folate, zinc, and optionally polyunsaturated fatty acids may be in one dosage unit and an additional ingredient, such as calcium, in a separate dosage unit.

A. Excipients

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Formulations are prepared using pharmaceutically acceptable “carriers” composed of materials that are considered safe and effective and may be administered to an individual without causing undesirable biological side effects or unwanted interactions. The term “carrier” refers to all components present in the pharmaceutical formulation other than the active ingredient or active ingredients. The term “carrier” includes but is not limited to diluents, binders, lubricants, disintegrators, fillers, and coating compositions. The term “carrier” also includes all components of the coating composition, which may include plasticizers, pigments, colorants, stabilizing agents, and glidants.

Optional pharmaceutically acceptable excipients present in the drug-containing tablets, capsules, beads, granules or particles include, but are not limited to, diluents, binders, lubricants, disintegrants, colorants, stabilizers, and surfactants. Diluents, also referred to as “fillers”, are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER®401, stearoyl monoisopropanolamide, and polyoxyethylene hydrogenated tallow amide. Examples of amphoteric surfactants include sodium N-dodecyl-.beta.-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose. Also suitable are hydrophobic polymers such as polyesters and polyimides. It is known to those skilled in the art that these polymers may be blended with polyanhydrides to achieve compositions with different drug release profiles and mechanical strengths. Preferably, the polymers are bioerodable, with preferred molecular weights ranging from 1000 to 15,000 kDa, and most preferably 2000 to 5000 Da.

The compounds may be complexed with other agents as part of their being pharmaceutically formulated. The compositions may take the form of, for example, tablets or capsules prepared by conventional means with pharmaceutically acceptable excipients such as binding agents (e.g., acacia, methylcellulose, sodium carboxymethylcellulose, polyvinylpyrrolidone (Povidone), hydroxypropyl methylcellulose, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complex then may be formulated in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

B. Modified Release Formulations

Delayed release and extended release compositions can be prepared according to methods readily known in the art. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionally, the formulation is coated with a film of a polymer which is insoluble in the acid environment of the stomach and soluble in the basic environment of lower GI tract in order to obtain a final dosage form that releases less than 10% of the drug dose within the stomach

Examples of rate controlling polymers that may be used in the dosage form are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT NE 30D (supplied by Rohm America). Gastrosoluble polymers, such as EUDRAGIT E100 or enteric polymers such as EUDRAGIT® RL100-55D, L100 and S100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

The delayed release dosage formulations may be prepared as described in references such as “Pharmaceutical dosage form tablets”, Eds. Liberman et al. (New York, Marcel Dekker, Inc., 1989), “Remington—The science and practice of pharmacy”, 20th Ed., Lippincott (Williams & Wilkins, Baltimore, Md., 2000), and “Pharmaceutical dosage forms and drug delivery systems”, 6th Ed., Ansel et al., (Media, Pa.: Williams and Wilkins, 1995) which provides information on carriers, materials, equipment and process for preparing tablets and capsules and delayed release dosage forms of tablets, capsules, and granules.

Tablets or capsules may be prepared containing the ingredients listed below in Table 1

TABLE 1
List of Ingredients and Amounts for Tablets or Capsules

| Ingredient | Amount | |
|---|---|---|
| Reduced folate (e.g. .e.5-methyl-(6S)- | 400-7,000 | μg |
| tetrahydrofolic acid) | | |
| Isoflavone (e.g. genistein) | 1-500 | mg |
| Zinc (e.g. zinc sulfate) | 5-100 | mg |
| Folic Acid | 50-60,000 | μg |
| Calcium | 20-2500 | mg |
| Vitamin D₃ | 1-2,000 | IU |
| Vitamin B₆ | 0.1-200 | mg |
| Vitamin B₁₂ | 2-250 | μg |
| Magnesium | 5-400 | mg |

Alternatively, tablets or capsules may be prepared containing the ingredients listed below in Table 2.

TABLE 2
List of Ingredients and Amounts for Tablets or Capsules

| Ingredient | Concentration | |
|---|---|---|
| Reduced folate (e.g. .e.5-methyl-(6S)- | 400-7,000 | μg |
| tetrahydrofolic acid) | | |
| Folic Acid | 50-60,000 | μg |
| Isoflavone (e.g. genistein) | 1-500 | mg |
| Zinc (e.g. zinc sulfate) | 5-100 | mg |
| Polyunsaturated Fatty Acids | 100-1,000 | mg |
| Calcium | 20-2500 | mg |
| Vitamin D₃ | 1-2,000 | IU |
| Vitamin B₆ | 0.1-200 | mg |
| Vitamin B₁₂ | 2-250 | μg |
| Magnesium | 5-400 | mg |

### III. Methods of Use

The compositions may be administered to treat all forms of osteoporosis including primary osteoporosis (juvenile osteoporosis, postmenopausal (Type I) osteoporosis and age-associated or senile (Type II) osteoporosis) and secondary osteoporosis (osteoporosis caused by chronic conditions and diseases such as, metabolic disease, connective tissue disease, bone marrow disease, immobilization, and drug use). In addition, the compositions may be administered for the prevention of osteoporosis in subjects who do not yet have the disease, but are at risk for getting osteoporosis, such as such as post-menopausal women, patients with osteopenia (mid thinning of the bone mass), or people who are over the age of 70. The compositions may also be administered to treat osteoporosis and/or inflammation in patients affected with inflammatory joint diseases such as rheumatoid arthritis (RA), Juvenile Rheumatoid Arthritis (JRA), psoriatic arthritis, Reiter's syndrome (reactive arthritis), Crohn's disease, ulcerative colitis, sarcoidosis.

Furthermore, the compositions may be administered to treat or prevent osteoporosis and/or inflammatory joint diseases in men and women with a folate deficiency or a folic acid metabolic disorder.

A. Disorders to be Treated

i. Osteoporosis

In one embodiment, the compositions may be administered to treat patients with all forms of osteoporosis. Osteoporosis is a systemic skeletal disease characterized by low bone mass and microarchitectural deterioration of bone tissue, with a consequent increase in bone fragility. Consequently, many individuals, both male and female, experience pain, disability, and diminished quality of life (QOL) caused by osteoporosis. The World Health Organization (WHO) has established the following definitions of osteoporosis based on bone mass density measurements in white women: Normal—Bone density no lower than 1 standard deviation (SD) below the mean for young adult women (T-score above-1); Low bone mass (osteopenia)—Bone density 1.0-2.5 SD below the mean for young adult women (T-score between −1 and −2.5); Osteoporosis—Bone density 2.5 SD or more below the normal mean for young adult females (T-score at or below −2.5).

Osteoporosis has been divided into several classifications according to etiology and localization in the skeleton. Osteoporosis initially is divided into localized and generalized categories. These main categories are classified further into primary and secondary osteoporosis.

Primary osteoporosis occurs in patients in whom a secondary cause of osteoporosis cannot be identified, including juvenile and idiopathic (type I and type II) osteoporosis. Juvenile osteoporosis usually occurs in children or young adults (approx 8-14 years old) of both sexes. These patients have normal gonadal function. The first sign of juvenile osteoporosis is usually pain in the lower back, hips, and feet, often accompanied by difficulty walking. There may also be knee and ankle pain and fractures of the lower extremities. Physical malformations also may be present. These include abnormal curvature of the upper spine (kyphosis), loss of height, a sunken chest, or a limp. These physical malformations are sometimes reversible after the juvenile osteoporosis has run its course. Type I osteoporosis (postmenopausal osteoporosis) usually occurs in women aged 50-65 years. This type of osteoporosis is characterized by a phase of accelerated bone loss, primarily from trabecular bone. In this phase, fractures of the distal forearm and vertebral bodies are common. Type II osteoporosis (age-associated or senile) occurs in both women and men older than 70 years. This form of osteoporosis represents bone loss associated with aging. Fractures comprise both cortical and trabecular bone. In addition to wrist and vertebral fractures, hip fractures often are seen in type II osteoporosis.

Secondary osteoporosis occurs when an underlying disease or chronic condition causes osteoporosis. This includes endogenous and exogenous thyroxine excess, hyperparathyroidism, malignancies, gastrointestinal diseases, medications, renal failure and connective tissue diseases, bone marrow disease, immobilization, and drug use. Even the clinical history may not be completely revealing, as a patient with known metastatic disease can develop compression fractures from osteoporosis secondary to chemotherapy or administration of steroids, and radiation therapy can weaken the bone.

In another embodiment, the compositions may be used to treat subjects who do not yet have osteoporosis, but who are at risk for getting osteoporosis, such as post-menopausal women, patients with osteopenia (mild thinning of the bone mass), subjects with chronic inflammatory joint diseases (described below) or people who are over the age of 70. Osteopenia results when the formation of bone (osteoid synthesis) is not enough to offset normal bone loss (bone lysis). Osteopenia is generally considered the first step along the road to osteoporosis. Diminished bone calcification, as seen on plain X-ray film, is referred to as osteopenia, whether or not osteoporosis is present. The diagnosis of osteopenia may also be made by a special X-ray machine for bone density testing. Other risk factors for osteoporosis, such as age and bone density, have been established by virtue of their direct and strong relationship to incidence of fractures; however, many other factors have been considered risk factors based on their relationship to bone density as a surrogate indicator of osteoporosis. Risk factors include the following: advanced age, female sex, white race, Asian ethnicity, family history of osteoporosis, small body frame, amenorrhea, late menarche, early menopause, null parity, physical inactivity, alcohol and tobacco use, androgen or estrogen deficiency, and calcium deficiency.

ii. Inflammatory Joint Diseases

In another embodiment, the compositions may be administered to treat patients affected with inflammatory joint diseases, with complications that include bone loss, fracture, and osteoporosis. For example, studies have found an increased risk of bone loss and fracture in individuals with rheumatoid arthritis and juvenile rheumatoid arthritis. People with these diseases are at increased risk for osteoporosis for many reasons: (1) glucocorticoid (corticosteroid) medications such as prednisone often prescribed for the treatment of rheumatoid arthritis or juvenile rheumatoid arthritis can trigger significant bone loss; (2) pain and loss of joint function caused by the diseases can result in inactivity, further increasing osteoporosis risk; (3) studies also show that bone loss in rheumatoid arthritis may occur as a direct result of the disease. The bone loss is most pronounced in areas immediately surrounding the affected joints. Other known diseases with similar complications that may be treated with the compositions include psoriatic arthritis, Reiter's syndrome (reactive arthritis), Crohn's disease, ulcerative colitis, and sarcoidosis. The compositions can be administered. in an effective amount, to (1) patients who have one or more chronic inflammatory joint diseases along with bone loss, fracture or osteoporosis or (2) patients who have one or more chronic inflammatory joint diseases and who do not have bone loss, fracture, or osteoporosis, but are at risk for these complications.

B. Administration Protocol

The compositions described herein may be administered one or more times during a 24-hour time period. For example, the compositions may be administered as a single dose of one or more tablets or capsules during a 24-hour time period. In a preferred embodiment, the compositions are administered in a once daily dose.

An intermittent administration protocol may be used, for example, where chronic administration is not desirable. The compound or formulation is administered in time blocks of several days with a defined minimum washout time between blocks. Typically, intermittent administration occurs over a period of several weeks to months to years to achieve a significant improvement in the symptoms of osteoporosis and chronic joint inflammation.

The compounds can be administered for a specific duration to improve symptoms of osteoporosis and/or chronic joint inflammation. A suitable endpoint can be where one symptom of the disorder is treated by administration of the compound and the treatment considered effective. In other situations, the treatment can be considered effective when more than one symptom is treated. In still other situations, the compositions may need to be administered to the patient for the duration of his or her life to prevent or treat osteoporosis and/or inflammatory joint disease, such as those patients with senile osteoporosis. The compositions may be modified in dosage as required by one skilled in the art. In one embodiment, the dosage can be modified by one skilled in the art to treat or prevent a disease or disorder, or lessen the risks associated with osteoporosis and or joint inflammation.

Compositions incorporating the above formulation are prepared using conventional methods and materials known in the pharmaceutical art.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

We claim:

1. A composition for the treatment or prevention of osteoporosis or inflammatory joint disease comprising one or more isoflavones and one or more reduced folates.

2. A composition for the treatment or prevention of osteoporosis or inflammatory joint disease comprising one or more isoflavones, one or more folates, and one or more polyunsaturated fatty acids.

3. The composition of claim 1, wherein the isoflavone is selected from the group consisting of daidzein, genistein, biochanin A, formononctin and glycitein.

4. The composition of claim 2, wherein the one or more folates comprises folic acid, one or more reduced folates, or a combination of folic acid and one or more reduced folates.

5. The composition of claim 1, wherein the one or more reduced folates are selected from the group consisting of 5-methyl-(6S)-tetrahydrofolic acid and 5-methyl-(6R,S)-tetrahydrofolic acid.

6. The composition of claim 1, wherein the composition further comprises zinc, calcium, or combinations thereof.

7. The composition of claim 1, further comprising one or more ingredients selected from the group consisting of vitamins, minerals, and emollient laxatives.

8. The composition of claim 1, wherein the composition is administered using one or more dosage forms.

9. A method for the treatment or prevention of osteoporosis in a patient in need thereof comprising administering a therapeutically effective amount of the composition of claim 1.

10. The method of claim 9, wherein the one or more isoflavones and or the one or more reduced folates are administered in the same dosage unit.

11. The method of claim 9, wherein the one or more isoflavones, the one or more folates, and the one or more polyunsaturated fatty acids are administered in the same dosage unit.

12. The method of claim 9, wherein the calcium is administered in a separate dosage form.

13. The method of claim 9, wherein the patient has a folic acid metabolism deficiency.

14. The method of claim 9, wherein the patient is selected from the group consisting of a post-menopausal woman, a subject with osteopenia, a subject with chronic inflammatory joint disease, and a patient over the age of 70.

15. A method for treating a chronic inflammatory joint disease in a patient in need thereof comprising administering a therapeutically effective amount of the composition of claim 1.

16. The method of claim 15, wherein the chronic inflammatory joint disease is selected from the group consisting of rheumatoid arthritis, juvenile rheumatoid arthritis, psoriatic arthritis, Reiter's syndrome, Crohn's disease, ulcerative colitis, and sarcoidosis.
